(19) United States
(12) Patent Application Publication
(10) Pub. No.: US 2026/0157727 A1
(43) Pub. Date: Jun. 11, 2026

(54) PROBE FALL PREVENTION ACCESSORIES

(71) Applicant: GE Precision Healthcare LLC, Waukesha, WI (US)

(72) Inventor: Mizuki Toyosato, Hino (JP)

(21) Appl. No.: 19/406,611

(22) Filed: Dec. 2, 2025

(30) Foreign Application Priority Data
Dec. 6, 2024 (JP) 2024-214059

(51) Int. Cl. A61B 8/00

(52) U.S. Cl. CPC A61B 8/44; A61B 8/467; A61B 8/54

(57) ABSTRACT

An article configured to be worn by an operator of an ultrasonic diagnostic device, the article preventing an ultrasonic probe of the ultrasonic diagnostic device from falling, the article including a detection unit that detects whether the article is worn by the operator; wherein, when the detection unit detects that the article is worn by the operator, the ultrasonic diagnostic device is set so as to be able to perform a prescribed operation.

### CROSS-REFERENCE TO RELATED APPLICATION

This application claim priority to Japanese Patent Application No. 2024-214059, which was file on Dec. 6, 2024 at the Japanese Patent Office. The entire contents of the above-listed application are incorporated by reference herein in their entirety.

### TECHINICAL FIELD

The present disclosure relates to an article for preventing an ultrasound probe from falling, and an ultrasound diagnostic system equipped with the article.

### BACKGROUND

Ultrasound diagnostic devices are known as devices for non-invasively imaging a subject. An ultrasonic diagnostic device is equipped with an ultrasonic probe including a transducer, and when scanning a subject, an operator holds the probe and scans the region of the patient to be imaged while operating the probe. Thereby, an ultrasound image of the patient's imaging area can be obtained.

### SUMMARY

A first aspect of the present disclosure is an article configured to be worn by an operator of an ultrasonic diagnostic device, the article preventing an ultrasonic probe of the ultrasonic diagnostic device from falling,
the article comprising:
a detection unit that detects whether the article is worn by the operator;
wherein, when the detection unit detects that the article is worn by the operator, the ultrasonic diagnostic device is set so as to be able to perform a prescribed operation.

A second aspect of the present disclosure is an ultrasonic diagnostic system having a function of preventing the ultrasonic probe from falling, the ultrasonic diagnostic system comprising:
an ultrasonic diagnostic device including an ultrasonic probe; and
an article configured to be worn by an operator of the ultrasonic diagnostic device and preventing the ultrasonic probe from being dropped, the article having a detection unit that detects whether the article is being worn by the operator, wherein
when the detection unit detects that the article is worn by the operator, the ultrasonic diagnostic device is set so as to be able to execute a prescribed operation.

### BRIEF DESCRIPTION OF THE DRAWINGS

FIG. 1 is a diagram depicting a state in which a subject 57 is being scanned using an ultrasound diagnostic system 100 according to an embodiment;

FIG. 2 is a block diagram of the ultrasound diagnostic system 100 according to an embodiment;

FIG. 3 is an explanatory diagram of an article 20 according to an embodiment;

FIG. 4 is an explanatory diagram of a wristband 21 according to an embodiment;

FIG. 5 is an explanatory diagram of a detection unit according to an embodiment;

FIG. 6 is a diagram depicting a state in which a detection unit 23 does not output a signal S1 indicating that the wristband 21 is worn on the wrist of an operator 56 according to an embodiment;

FIG. 7 is a diagram depicting the operation flow of an ultrasound diagnostic device 1 according to an embodiment;

FIG. 8 is a diagram depicting an example of a warning to prompt the operator 56 to wear the wristband 21 according to an embodiment;

FIG. 9 is an explanatory diagram of a detection unit 23 according to an embodiment and

FIG. 10 is a diagram depicting a state in which the detection unit 23 does not output a signal S2 indicating that the wristband 21 is not attached to the wrist of the operator 56 according to an embodiment.

### DETAILED DESCRIPTION

The vibrator is very thin and may be damaged if the operator drops the ultrasound probe while scanning a patient. Therefore, in order to prevent the ultrasound probe from falling while the operator is scanning the patient, there is known an item that connects the cable of the ultrasound probe to the operator's wrist.

The article includes a wristband that can be worn on the wrist of an operator and a strap for connecting the wristband to the ultrasound probe (or the cable of the ultrasound probe). Use of this article can prevent the probe from falling, even if the operator accidentally lets go of the probe while scanning a patient, thereby avoiding damage to the probe.

However, when examining a patient, the operator may forget to wear the article for preventing falling while scanning the patient. In this case, scanning of the patient is executed without using any fall prevention items, so if the operator accidentally lets go of the probe while scanning the patient, the probe may fall and be damaged.

Therefore, there is a demand for a technology that can improve the situation in which an operator scans a subject without wearing the article to prevent the subject from falling.

In the present disclosure, a detection unit is provided that detects whether an item for preventing the ultrasound probe from falling is worn by the operator, and when it is detected that this item is worn by the operator, the ultrasound diagnostic device can execute a prescribed operation. Therefore, unless the operator puts on an article to prevent the ultrasound probe from falling, the ultrasound diagnostic device will not execute operation, so the operator can be made to put on the article to prevent the ultrasound probe from falling before scanning the subject. This prevents the operator from scanning the subject without wearing the article to prevent the ultrasound probe from falling, thereby avoiding the risk of the probe falling and being damaged while scanning the subject.

Embodiments will be described below, but the present disclosure is not limited to the following embodiments.

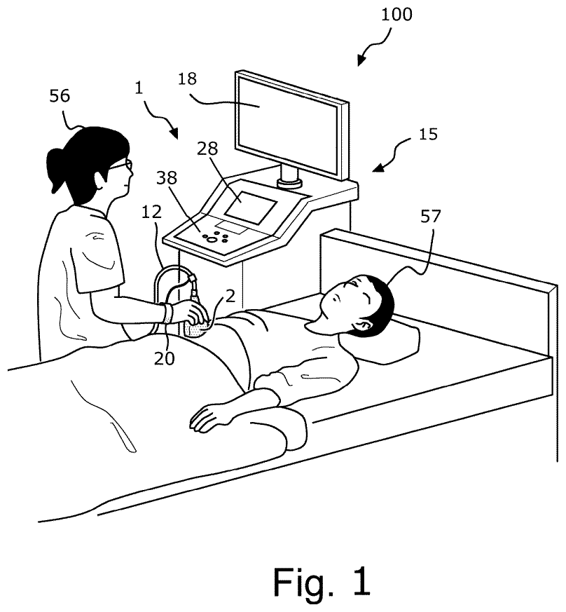

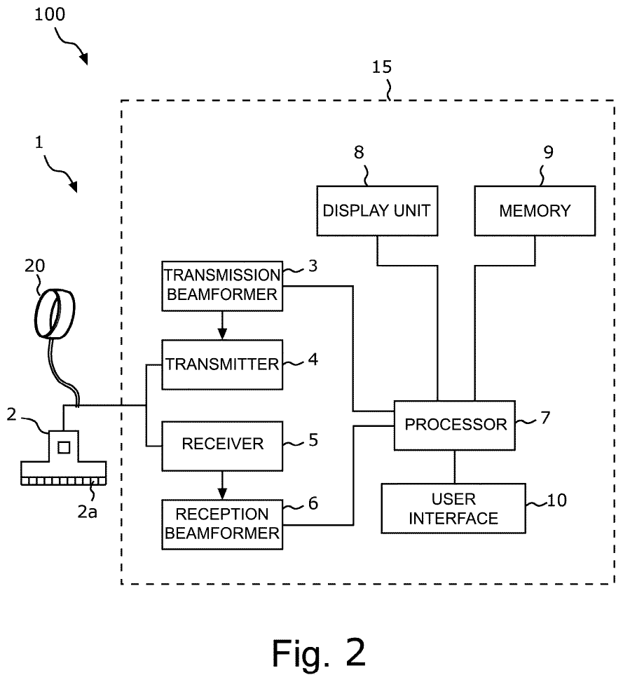

FIG. 1 is a diagram depicting a state in which a subject 57 is being scanned using an ultrasound diagnostic system 100 according to the present embodiment. FIG. 2 is a block diagram of the ultrasound diagnostic system 100.

The ultrasound diagnostic system 100 includes an ultrasound diagnostic device 1 and an article 20. The article 20 is an article for preventing the ultrasonic probe from falling. The article 20 will be described later.

The ultrasonic diagnostic device 1 includes a device main body part 15 and an ultrasonic probe 2. The device main body part 15 includes a transmit beamformer 3, a transmitter 4, a receiver 5, a receive beamformer 6, a processor 7, a display part 8, memory 9, and a user interface 10.

The ultrasonic probe 2 has a plurality of vibrating elements 2a arranged in an array. The transmit beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating element 2a are reflected in the subject 57 (see FIG. 1) and a reflection echo is received by the vibrating element 2a. The vibrating elements 2a convert the received echo to an electrical signal and output this electrical signal as an echo signal to the receiver 5. The receiver 5 executes a prescribed process on the echo signal and outputs the echo signal to the receive beamformer 6. The receive beamformer 6 executes reception beamforming on the signal received through the receiver 5 and outputs echo data.

The receive beamformer 6 may be a hardware beamformer or a software beamformer. If the receive beamformer 6 is a software beamformer, the receive beamformer 6 may include one or a plurality of processors, including one or a plurality of: i) a graphics processing unit (GPU); ii) a microprocessor; iii) a central processing unit (CPU); iv) a digital signal processor (DSP); or v) another type of processor capable of executing logical operations. A processor configuring the receive beamformer 6 may be configured by a processor different from the processor 7 or may be configured by the processor 7.

The ultrasonic probe 2 may include an electrical circuit for performing all or a portion of transmission beamforming and/or reception beamforming. For example, some or all of the transmit beamformer 3, the transmitter 4, the receiver 5, and the receive beamformer 6 may be provided in the ultrasonic probe 2.

The processor 7 controls the transmit beamformer 3, the transmitter 4, the receiver 5, and the receive beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display part 8. The processor 7 can process echo data to generate an ultrasound image. The term “electronic communication” may be defined to include both wired and wireless communications. The processor 7 may include a central processing unit (CPU) according to one embodiment. According to another embodiment, the processor 7 may include another electronic component that may perform a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not depicted) that demodulates RF data. In another embodiment, demodulation may be executed in an earlier step in the processing chain.

Furthermore, the processor 7 may generate various ultrasound images (e.g., a B-mode image, color Doppler image, M-mode image, color M-mode image, spectral Doppler image, elastography image, TVI image, strain image, strain rate image, and the like) on the basis of data obtained by processing via the receive beamformer 6. In addition, one or a plurality of modules can generate these ultrasound images.

An image beam and/or an image frame may be saved and timing information may be recorded indicating when the data is retrieved to the memory. The module may include, for example, a scan conversion module that executes a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasound images may be read from the image memory and displayed on the display part 8.

In the present Specification, the term “image” can broadly indicate both a visual image and data representing a visual image. Furthermore, the term “data” can include raw data, which is ultrasound data before a scan conversion operation, and image data, which is data after the scan conversion operation.

Note that the processing tasks described above handled by the processor 7 may be executed by a plurality of processors.

Furthermore, when the receive beamformer 6 is a software beamformer, a process executed by the beamformer may be executed by a single processor or may be executed by the plurality of processors.

Examples of the display part 8 include a LED (Light-Emitting Diode) display part, an LCD (Liquid Crystal Display), and an organic EL (Electro-Luminescence) display part. The display part 8 displays an ultrasound image. In an embodiment, the display part 8 includes a display monitor 18 and a touch panel 28, as depicted in FIG. 1. However, the display part 8 may be configured of a single display part rather than the display monitor 18 and the touch panel 28. Moreover, two or more display devices may be provided in place of the display monitor 18 and the touch panel 28.

The memory 9 is any known data storing medium. In one example, the ultrasound image display system includes a non-transitory storing medium and a transitory storing medium as memories. In addition, the ultrasound image display system may also include a plurality of memories. The non-transitory storing medium is, for example, a non-volatile storing medium such as an HDD (Hard Disk Drive: hard disk drive), a ROM (Read-Only Memory), or the like. The non-transitory storing medium may include a portable storing medium such as a CD (Compact Disk), a DVD (Digital Versatile Disk), or the like. A program executed by the processor 7 is stored in the non-transitory storing medium. The transitory storing medium is a volatile storing medium such as a Random-Access Memory (RAM) or the like.

The memory 9 stores one or a plurality of instructions that can be executed by the processor 7.

The processor 7 may also be configured to be able to connect to an external storage device (not depicted) via a wired or wireless connection. In this case, the instruction causing execution by the processor 7 can be distributed to both the memory 9 and the external storing device for storage.

The user interface 10 may accept input from an operator 56. For example, the user interface 10 accepts commands and information input from an operator 56. The user interface 10 may include a touch panel 28 and an operation panel 38. The operation panel 38 includes a keyboard, a hard key, a trackball, a rotary controller, and the like. The touch panel 28 can also display soft keys, buttons, and the like.

The ultrasound diagnostic system 100 is also provided with an article 20 for preventing the probe 2 from falling.

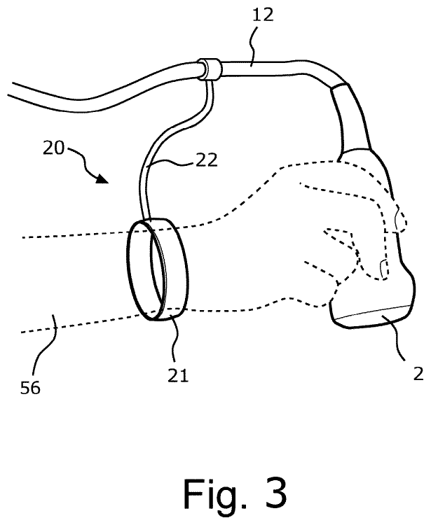

FIG. 3 is an explanatory diagram of the article 20.

The article 20 includes a wristband 21 that can be worn on the wrist of the operator 56 and a strap 22 for connecting the wristband 21 to a probe cable 12 of the ultrasonic probe 2.

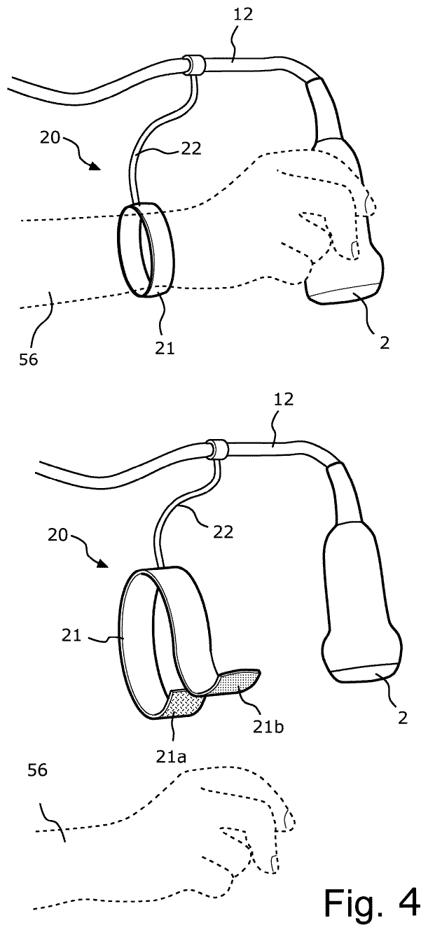

FIG. 4 is an explanatory diagram of the wristband 21.

The upper part of FIG. 4 depicts the wristband 21 in a state where the wristband is attached to the wrist of the operator 56, and the lower part of FIG. 4 depicts the wristband 21 in a state where the wristband has been removed from the wrist of the operator 56.

The wristband 21 is configured so that a front surface 21a and a back surface 21b of the wristband 21 can be attached and detached to each other. The front surface 21a and the back surface 21b may be, for example, hook-and-loop fasteners. The wristband 21 may also be configured as a binding band to enable being attached to the operator 56. When putting on the wristband 21, the operator 56 peels the back surface 21b of the wristband 21 from the front surface 21a of the wristband 21, wraps the wristband 21 around their wrist, and presses the back surface 21b of the wristband 21 against the front surface 21a, thereby putting on the wristband 21. Then, by peeling the back surface 21b of wristband 21 from the front surface 21a, the wristband 21 can be removed from the wrist. In this manner, the wristband 21 is configured so that the state of the wristband 21 can be switched between a first state in which the back surface 21b is attached to the front surface 21a, and a second state in which the back surface 21b is separated from the front surface 21a.

By wearing the wristband 21 on the wrist, even if the operator 56 accidentally lets go of the probe 2 while scanning the subject, the probe 2 is prevented from dropping, and damage to the probe 2 can be avoided.

However, when examining a patient, the operator 56 may forget to put on the fall prevention article 20 and scan the subject. In this case, scanning of the subject is executed without using the fall prevention article 20, so if the operator 56 suddenly lets go of the probe 2 while scanning the subject, the probe 2 may fall and cause damage to the probe 2.

In the present embodiment, in order to address this problem, the article 20 is provided with a detection unit that detects whether the operator 56 has put on the wristband 21. This detection unit will be described below.

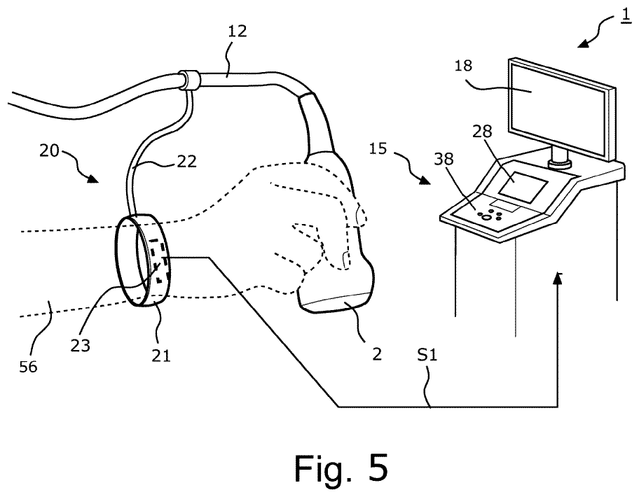

FIG. 5 is an explanatory diagram of the detection unit. The wristband 21 of the article 20 includes a built-in detection unit 23 that detects whether or not the wristband 21 is worn by the operator 56. When the back surface 21b of the wristband 21 is attached to the front surface 21a, the detection unit 23 detects that the wristband 21 is wrapped around the wrist of the operator 56 (in other words, detects that the wristband 21 is worn by the operator 56). When the wristband 21 is detected to have been put on, a signal S1 indicating that the wristband 21 has been put on the wrist of the operator 56 is output to the device main body part 15 of the ultrasonic diagnostic device 1. The signal S1 may be transmitted from the article 20 to the device main body part 15 via the probe cable 12, or may be wirelessly transmitted from the article 20 to the device main body part 15. When the device main body part 15 receives this signal S1, the processor 7 of the device main body part 15 sets the ultrasound diagnostic device 1 to an authorized mode in which scanning of the subject is authorized. When the ultrasound diagnostic device 1 is set to the authorized mode, when the operator 56 operates the ultrasound diagnostic device 1 to execute a scan of the subject, the ultrasound diagnostic device 1 can execute an operation corresponding to the operation of the operator 56. Therefore, the subject can be scanned.

The detection unit 23 can be configured by, for example, a capacitance touch sensor and/or an optical sensor. As an optical sensor, for example, a heart rate sensor can be used. These sensors may be supplied with power through the probe cable 12, or a battery may be installed in the wristband 21 and power may be supplied to the sensors from this battery. The detection unit 23 may also be provided with a button switch that detects whether the front surface 21a and the back surface 21b of the wristband 21 are attached to each other, and whether the wristband 21 is worn may be detected by switching the button switch on and off. A power-off switch can be used as the button switch.

In FIG. 5, the wristband 21 is attached to the wrist of the operator 56, so that even if the operator 56 suddenly lets go of the probe 2 while scanning the subject, the probe 2 is prevented from falling and damage to the probe 2 can be avoided.

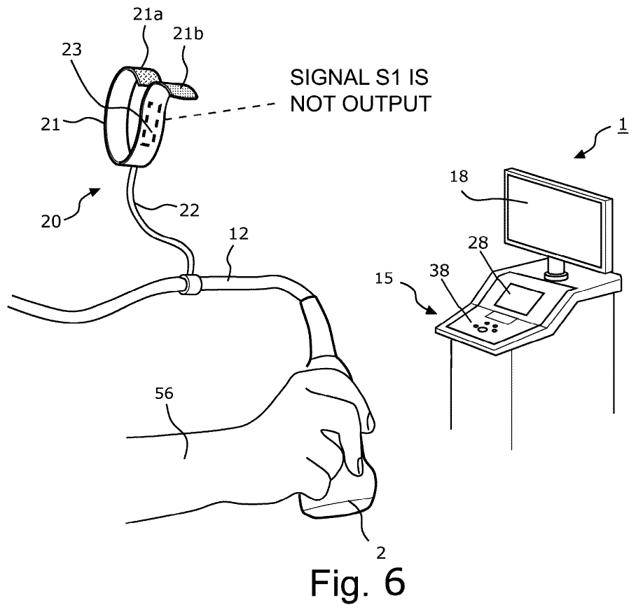

Next, the operation of the detection unit 23 when the operator 56 is not wearing the wristband 21 will be described with reference to FIG. 6.

If the back surface 21b of the wristband 21 is separated from the front surface 21a, the detection unit 23 detects that the wristband 21 is not wrapped around the wrist of the operator 56 (in other words, detects that the wristband 21 is not worn by the operator 56). In this case, the detection unit 23 does not output a signal S1 indicating that the wristband 21 is attached to the wrist of the operator 56, so the processor 7 sets the ultrasound diagnostic device 1 to a non-authorized mode in which scanning of the subject is not authorized. When the ultrasound diagnostic device 1 is set to the non-authorized mode, even if the operator 56 operates the ultrasound diagnostic device 1 to execute a scan of the subject, the ultrasound diagnostic device 1 is set to not execute the operation corresponding to the operation of the operator 56. Therefore, in the non-authorized mode, the subject cannot be scanned.

In the non-authorized mode, the ultrasound diagnostic device 1 may be set so that all operations cannot be executed, or so that some operations cannot be executed. For example, in the non-authorized mode, the ultrasound diagnostic device 1 may be set so that the various buttons on the operation panel 38 do not function, but the various soft switches on the touch panel 28 do function. In addition, in the non-authorized mode, the ultrasound diagnostic device 1 may be set so that the various soft switches on the touch panel 28 do not function, but the various buttons on the operation panel 38 do function. Furthermore, some of the buttons on the operation panel 38 may be set to function, while the remaining buttons may not function. Similarly, some of the soft switches on the touch panel 28 may be set to function, while the remaining soft switches may not function.

As described above, when the operator 56 is not wearing the wristband 21, even if the operator 56 operates the ultrasound diagnostic device 1, the ultrasound diagnostic device 1 will not execute the operation corresponding to the operator's operation, and the operator will not be able to scan the subject. Therefore, the operator 56 recognizes that images of the subject cannot be captured without the wristband 21 attached, and therefore attaches the wristband 21 to the wrist in order to capture images of the subject. When the wristband 21 is attached to the wrist of the operator 56, the detection unit 23 outputs a signal S1 to the device main body part 15 of the ultrasound diagnostic device 1, as depicted in FIG. 5, indicating that the wristband 21 is attached to the wrist of the operator 56. Therefore, the ultrasonic diagnostic device 1 is set to authorized mode in which scanning of the subject is authorized, and the operator can scan the subject.

As described above, if the wristband 21 is not attached to the wrist of the operator 56, even if the operator 56 operates the ultrasound diagnostic device 1, the ultrasound diagnostic device 1 will not execute an operation in accordance with the operation of the operator 56. Therefore, if the wristband 21 is not attached to the wrist of the operator 56, the operator 56 cannot proceed to the step of scanning the subject, so scanning the subject without the operator 56 wearing the wristband 21 can be avoided. When the operator 56 is detected to be wearing the wristband 21 on the wrist, the ultrasonic diagnostic device 1 is set to authorized mode in which scanning of the subject is permitted, enabling the operator 56 to scan the subject. Accordingly, the operator 56 can be reliably caused to wear the wristband 21 when scanning the subject, which reliably prevents the probe 2 from being dropped during scanning.

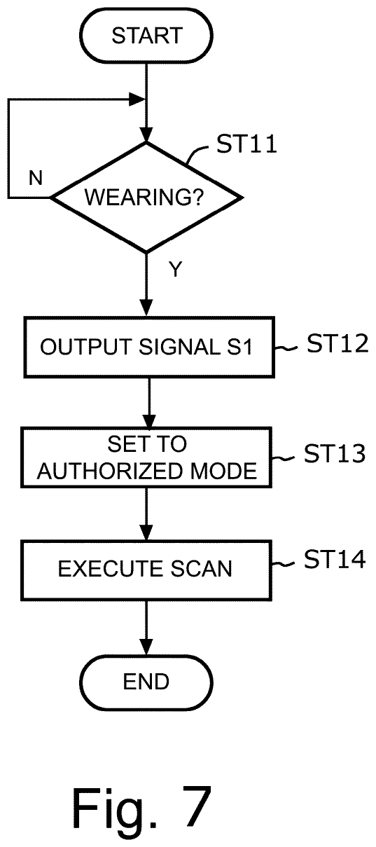

Next, the operational flow of the ultrasonic diagnostic device 1 when capturing an image of a subject using the article 20 configured as described above will be described with reference to the flowchart of FIG. 7.

Step ST11 determines whether the detection unit 23 of the article 20 detects that the wristband 21 is worn by the operator 56.

For example, as depicted in FIG. 6, when the operator 56 is not wearing the wristband 21, the detection unit 23 does not output a signal S1 indicating that the wristband 21 is worn on the wrist of the operator 56. Therefore, the ultrasound diagnostic device 1 is set to a non-authorized mode in which scanning of the subject is not authorized. In this case, even if the operator 56 operates the ultrasound diagnostic device 1, they are not able to scan the subject. Accordingly, if the wristband 21 is not worn on the wrist of the operator 56, the operator 56 can be prevented from scanning the subject while not wearing the wristband 21.

On the other hand, as depicted in FIG. 5, when the operator 56 puts on the wristband 21, the detection unit 23 detects that the wristband 21 is worn by the operator 56. The process then proceeds to step ST12.

In step ST12, the detection unit 23 outputs a signal S1 indicating that the wristband 21 is being worn on the wrist of the operator 56 to the device main body part 15 of the ultrasound diagnostic device 1. When the signal S1 is output, the process proceeds to step ST13.

In step ST13, in response to signal S1, the processor of the ultrasound diagnostic device 1 switches the ultrasound diagnostic device 1 from the non-authorized mode in which scanning of the subject is not authorized to an authorized mode in which scanning of the subject is authorized. By switching the ultrasonic diagnostic device 1 to the authorized mode, the ultrasonic diagnostic device 1 can execute an operation for scanning the subject in response to an operation by the operator 56. After the ultrasonic diagnostic device 1 switches to the authorized mode, the process proceeds to step ST14, where the operator 56 executes scanning of the subject, and the flow ends.

In the present embodiment, if the wristband 21 is not worn on the wrist of the operator 56, even if the operator 56 operates the ultrasound diagnostic device 1 to execute scanning of the subject, the ultrasound diagnostic device 1 will not execute the operation corresponding to the operation of the operator 56. Therefore, if the wristband 21 is not worn on the wrist of the operator 56, the operator 56 cannot scan the subject, and therefore, the operator 56 is prevented from operating the probe 2 without wearing the wristband 21.

On the other hand, when the operator 56 puts on the wristband 21, a signal S1 indicating that the operator 56 has put on the wristband 21 is transmitted to the device main body part 15 of the ultrasound diagnostic device 1, and the ultrasound diagnostic device 1 is set to authorized mode in which scanning of the subject is authorized. Therefore, the operator 56 executes scanning of the subject while wearing the wristband 21, thereby avoiding the danger of the probe 2 falling and breaking down during scanning of the subject.

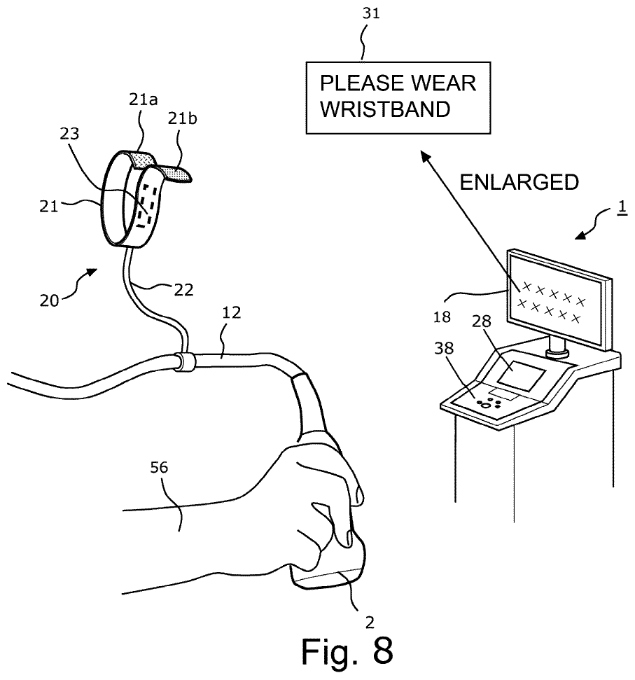

If the operator 56 is not wearing the wristband 21, the ultrasonic diagnostic device 1 may display a warning to prompt the operator 56 to wear the wristband 21 (see FIG. 8).

FIG. 8 is a diagram depicting an example of a warning to prompt the operator 56 to wear the wristband 21.

FIG. 8 depicts an enlarged view of a warning 31 displayed on the display part 8. The display part 8 displays the warning 31 indicating “Please wear the wristband 21”. Therefore, by looking at the display part 8, the operator 56 can visually recognize that the wristband 21 needs to be worn.

According to an embodiment, the operator 56 puts on the wristband 21 before starting to scan the subject. Then, the operator 56 can remove the wristband 21 after completing the scan of the subject. Therefore, by measuring the time that the operator 56 wears the wristband 21, the examination time of the subject can be measured, and therefore the examination time can be automatically managed.

According to an embodiment, the wristband 21 has a structure in which the back surface can be detachably attached to the front surface. However, the wristband 21 is not limited to this type of structure, and may have another structure as long as attaching to the operator 56 is feasible. For example, a stretchable wristband (such as a silicone band) having a closed loop shape may be used.

According to an embodiment, the article 20 is configured to be worn on the wrist of the operator. However, the article 20 is not limited to being worn on the wrist as long as preventing the probe from falling is possible. For example, a structured article that can be worn on the operator's finger or upper arm may be used to prevent the probe from falling.

According to an embodiment, the ultrasonic probe 2 is connected to the device main body part 15 by the probe cable 12. However, the ultrasonic probe 2 may be connected to the device main body part 15 wirelessly. In this case, the strap of the article 20 may be configured to be connected to the ultrasound probe 2.

According to an embodiment, when the detection unit 23 of the article 20 detects that the wristband 21 is worn on the wrist of the operator 56, a signal S1 indicating that the wristband 21 is worn on the wrist of the operator 56 is output to the device main body part 15 of the ultrasound diagnostic device 1, as depicted in FIG. 5.

In contrast, according to another embodiment, an example is described in which, when the wristband 21 is not worn by the operator 56, the detection unit 23 of the article 20 is configured to output a signal S2 indicating that the wristband 21 is not worn on the wrist of the operator 56.

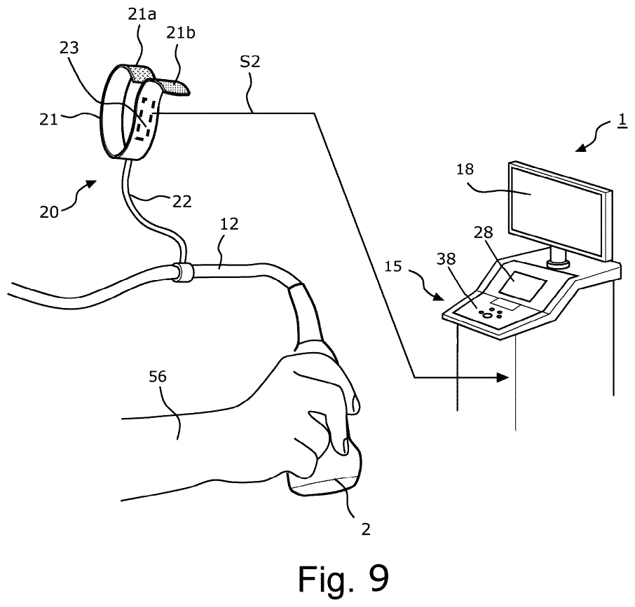

FIG. 9 is an explanatory diagram of the detection unit 23 according to an embodiment. When the detection unit 23 detects that the wristband 21 is not worn on the wrist of the operator 56, the unit outputs a signal S2 to the device main body part 15 of the ultrasound diagnostic device 1, indicating that the wristband 21 is not attached to the wrist of the operator 56. The signal S2 may be transmitted from the article 20 to the device main body part 15 via the probe cable 12, or may be wirelessly transmitted from the article 20 to the device main body part 15. When the device main body part 15 receives this signal S2, the processor of the device main body part 15 sets the ultrasound diagnostic device 1 to a non-authorized mode in which scanning of the subject is not authorized. When the ultrasonic diagnostic device 1 is set to non-authorized mode, the operator 56 cannot scan the subject even if he operates the ultrasonic diagnostic device 1. Accordingly, if the wristband 21 is not worn on the wrist of the operator 56, the operator 56 can be prevented from scanning the subject while not wearing the wristband 21.

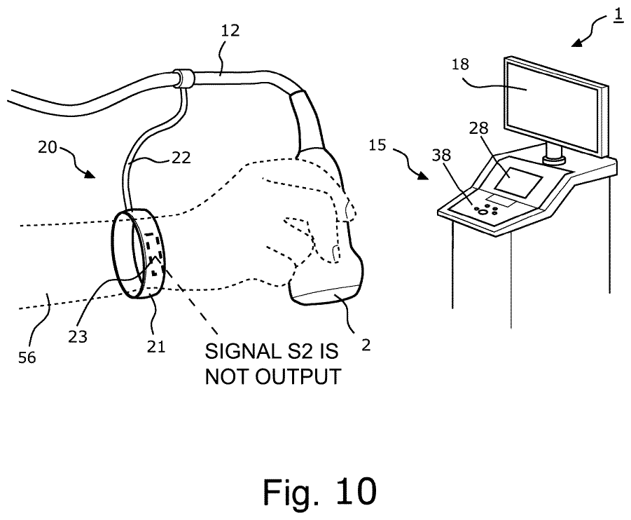

On the other hand, as depicted in FIG. 10, when the operator 56 puts on the wristband 21, the detection unit 23 does not output the signal S2 indicating that the wristband 21 is not worn on the wrist of the operator 56. In this case, since the ultrasonic diagnostic device 1 does not receive the signal S2, the processor of the ultrasonic diagnostic device 1 sets the ultrasonic diagnostic device 1 to an authorized mode in which scanning of the subject is authorized. Therefore, when the operator 56 operates the ultrasonic diagnostic device 1, the ultrasonic diagnostic device 1 executes an operation in accordance with the operation of the operator 56. In this case, the operator 56 can scan the subject while wearing the wristband 21 on his/her wrist, thereby avoiding the risk of the probe 2 falling.

According to an embodiment, when the wristband 21 is not worn by the operator 56, a signal S2 is output indicating that the article 20 is not worn on the wrist of the operator 56, and when the wristband 21 is worn by the operator 56, the signal S2 is not output. Therefore, whether the wristband 21 is worn by the operator 56 can be determined depending on whether the signal S2 is output. If the operator 56 is not wearing the wristband 21, the ultrasound diagnostic device 1 is set to non-authorized mode, and the device cannot proceed to the step of scanning the subject, thereby preventing the operator 56 from operating the probe 2 without wearing the wristband 21. On the other hand, when the operator 56 wears the wristband 21, the ultrasound diagnostic device 1 is set to authorized mode, and the operator 56 executed a scan of the subject while wearing the wristband 21. Therefore, even if the probe 2 is released from the hand during scanning, the probe 2 is prevented from falling, and damage to the probe 2 can be prevented.
1. Ultrasonic diagnostic device
2. Ultrasonic probe
2a. Vibrating element
3. Transmit beamformer
4. Transmitter
5. Receiver
6. Receive beamformer
7. Processor
8. Display part
9. Memory
10. User interface
12. Probe cable
15. Device main body part
18. Display monitor
20. Article
21. Wristband
22. Strap
21a. Front surface
21b. Back surface
23. Detecting unit
28. Touch panel
31. Warning
38. Operation panel
56. Operator
57. Subject
100. Ultrasound diagnostic system

What is claimed is:

1. An ultrasonic diagnostic device, comprising:
a probe comprising a transducer;
a processor;
a memory storing instructions;
an article configured to be worn by an operator of the ultrasonic diagnostic device, the article preventing an ultrasonic probe of the ultrasonic diagnostic device from falling, the article comprising a detection unit that detects whether the article is worn by the operator;
wherein, the processor is configured to perform a prescribed operation based on the detection unit detecting that the article is worn by the operator.

2. The article according to claim 1, wherein the detection unit is configured to output a first signal to a main body part of the ultrasonic diagnostic device when the article is worn by the operator, and when the main body part of the ultrasonic diagnostic device receives the first signal, the ultrasonic diagnostic device is set to be able to execute a prescribed operation.

3. The article according to claim 2, wherein when the ultrasonic diagnostic device receives the first signal, and the operator operates the ultrasonic diagnostic device, the ultrasonic diagnostic device executes an operation in response to the operator's operation.

4. The article according to claim 1, wherein the detection unit is configured to output a second signal to the main body part of the ultrasonic diagnostic device when the article is not worn by the operator, and when the main body part of the ultrasonic diagnostic device receives the second signal, the ultrasonic diagnostic device is set so as to be unable to execute the prescribed operation, and when the main body part of the ultrasonic diagnostic device does not receive the second signal, the ultrasonic diagnostic device is set so as to be able to execute the prescribed operation.

5. The article according to claim 4, wherein, when the main body part of the ultrasonic diagnostic device receives the second signal, the ultrasonic diagnostic device does not execute an operation corresponding to an operation by the operator even if the operator operates the ultrasonic diagnostic device, and when the main body part of the ultrasonic diagnostic device does not receive the second signal, the ultrasonic diagnostic device executes an operation corresponding to an operation by the operator when the operator executes the ultrasonic diagnostic device.

6. The article of claim 1, wherein the article comprises a wristband that can be worn around the subject's wrist.

7. The article according to claim 6, wherein the detection unit outputs the first signal to a main body part of the ultrasonic diagnostic device when the detection unit detects that the wristband is worn on the wrist of the operator.

8. The article according to claim 6, wherein the detection unit outputs the second signal to a main body part of the ultrasonic diagnostic device when the detection unit detects that the wristband is not worn on the wrist of the operator.

9. The article according to claim 6, wherein the detection unit includes a capacitive touch sensor and/or an optical sensor.

10. The article of claim 9, wherein the optical sensor is a heart rate sensor.

11. The article according to claim 6, wherein the wristband includes a first portion and a second portion that can be detachably attached to the first portion, and the wristband is configured so that the state of the wristband can be switched between a first state in which the second portion is attached to the first portion and a second state in which the second portion is separated from the first portion, and the detection unit detects that the wristband is wrapped around the wrist of the operator in the first state, and detects that the wristband is not wrapped around the wrist of the operator in the second state.

12. The article of claim 10, wherein the detection unit includes a switch for detecting whether the wristband is worn by an operator.

13. The article of claim 11, wherein the switch is a powerless switch.

14. An ultrasonic diagnostic system having a function of preventing the ultrasonic probe from falling, the ultrasonic diagnostic system comprising:
an ultrasonic diagnostic device including an ultrasonic probe; and
an article configured to be worn by an operator of the ultrasonic diagnostic device and preventing the ultrasonic probe from being dropped, the article having a detection unit that detects whether the article is being worn by the operator, wherein
when the detection unit detects that the article is worn by the operator, the ultrasonic diagnostic device is set to be able to execute a prescribed operation.

15. The ultrasonic diagnostic system according to claim 12, wherein if the article is not attached to the operator, the ultrasonic diagnostic device displays a warning to prompt the operator to wear the article.
